(19) United States
(12) Patent Application Publication
(10) Pub. No.: US 2007/0225370 A1
(43) Pub. Date: Sep. 27, 2007

(54) Nutritional Compositions and Methods

(76) Inventor: Joar Opheim, Aptos, CA (US)

Correspondence Address: FOOTHILL LAW GROUP, 777 N. FIRST STREET, SUITE 325, SAN JOSE, CA, 95112, US

(21) Appl. No.: 11/689,433

(22) Filed: Mar. 21, 2007

Related U.S. Application Data
(60) Provisional application No. 60/743,607, filed on Mar. 21, 2006.

(51) Int. Cl. A61K 31/202

(52) U.S. Cl. 514/560

(57) ABSTRACT

The invention is an anti-inflammatory composition comprising a mixture comprising two components, a first component rich in EPA and a second component rich in GLA. The first component is a mixture comprising EPA and preferably DHA comprising at least 35% EPA, preferably 50% EPA and more preferably 60% or more of EPA, and up to 25% DHA, preferably up to 10% DHA, possibly 0% DHA. The second component comprises at least 20% GLA, preferably 60%, and more preferably 70% GLA. The preferred composition comprises between about 60 and 80% of the first component and 20% and 40% of the second component.

This application claims the benefit of my Provisional Patent Application No. 60/743,607 filed on Mar. 21, 2006, which is hereby incorporated herein by reference.

### BACKGROUND OF THE INVENTION

1. Field of the Invention

This invention relates to nutritional compositions which when ingested will promote formation of anti-inflammatory substances in the human body in preference to pro-inflammatory substances. The compositions comprise components found in omega-3 and omega-6 oils which are refined to change the relative proportions of the components. The compositions will act as alternatives to NSAIDs as well as providing other benefits.

2. Description of the Prior Art

Omega-3 and Omega-6 fatty acids are well known in the literature to play a major role in human health. Dr. Joseph Maroon recently published a study entitled “Omega-3 Fatty acids (fish oil) as an anti-inflammatory: An alternative to non-steroidal anti-inflammatory drugs for discogenic pain” in the April, 2006 issue of Surgical Neurology. The study indicates that the anti-inflammatory properties of fish oils rich in Omega-3 fatty acids allowed 60% of patients to discontinue their use of NSAIDs. The importance of this work is to show that there is an alternative to NSAIDS which does not have the undesirable side effects of NSAIDs. Additional clinical trials have shown that arthritis patients who take fish oils could eliminate or sharply reduce their use of NSAIDs. The anti-inflammatory properties of Omega-3 fatty acid are generally attributed to the production of eicosanoids including prostaglandins, thromboxanes, and leukotrienes which are made in vivo by enzyme catalyzed reactions of eicosapentanoic acid (EPA) found in fish oil. The eicosanoids produced from Omega-3 fatty acids are all anti-inflammatory.

All eicosanoids are not anti-inflammatory, however. Eicosanoids made from Omega-6 fatty acids (found in vegetable oils such as corn, safflower, sunflower, evening primrose, and borage oils) contain both pro-inflammatory eicosanoids, made from Arachidonic Acid (AA), and also anti-inflammatory eicosanoids made from Dihomo-Gamma-Linolenic-Acid (DGLA). The anti-inflammatory eicosanoids made from Omega-6 fatty acids are not made from Omega-3 fatty acids.

There is a need for a combined Omega-3/Omega-6 supplement that produces the anti-inflammatory eicosanoids of from both Omega-3 and Omega-6 fatty acids while at the same time minimizing the production of the pro-inflammatory eicosanoids made through the Omega-6 AA pathway. Such a supplement will be even more effective than Omega-3 as an NSAID alternative.

### SUMMARY OF THE INVENTION

The invention deals with a new nutritional composition which promotes the formation of anti-inflammatory substances, particularly eicosanoids, from omega-3 and omega-6 fatty acids, in preference to pro-inflammatory eicosanoids. Eicosanoids include prostaglandins (PGE), thromboxanes (TXA), and leukotrienes (LTB). Each of these families of eicosanoids has both pro-inflammatory as well as anti-inflammatory members. Eicosanoids are made in vivo by enzyme catalyzed reactions involving omega-3 fatty acids in particular EPA and DHA (eicosapentaenoic and docosahexaenoic acid respectively). EPA and DHA are found in fish oil. EPA and DHA are also made in the body from Alpha-Linolenic Acid (ALA) which is found in fish oil and flaxseed oil. Eicosanoids are also made in vivo from Omega-6 Fatty Acids which are found in vegetable sources such as corn, sunflower and safflower oils as linoleic acid (LA), and gamma-linolenic acid (GLA). GLA is produced to a limited extent from LA in the body and is also found directly in evening primrose, borage, and black currant seed oils.

The eicosanoids made from Omega-3 oils are anti-inflammatory eicosanoids which derive from enzymatic reaction of EPA by cyclooxygenase (COX) and lipoxygenase (LOX) enzymes

Prostaglandins PGE-3 and leukotriene LTB-5, are anti-inflammatory eicosanoids, which are synthesized in the body from EPA, an omega-3 fatty acid. PGE-1 is an anti-inflammatory prostaglandin that is synthesized in the body from Dihomo-Gamma-Linolenic Acid (DGLA), an omega-6 fatty acid.

Leukotriene LTB-4, thromboxane TXA-2, and prostaglandin PGE-2 are pro-inflammatory eicosanoids which the body synthesizes from arachidonic acid (AA), an omega-6 fatty acid. LTB-4, TXA-2 and PGE-2 are made from COX and LOX catalyzed reaction of AA, and are known to increase sensitivity to pain, increase swelling, induce fever etc.

AA is made in vivo from DGLA by an enzymatic reaction of delta-5 desaturase.

It is known that addition of Eicosapentaenoic acid (EPA) to GLA inhibits delta-5-desaturase activity, the terminal enzymatic step in AA synthesis. (Barham J B, Edens M B, et al., Addition of Eicosapentaenoic Acid to Gamma-Linolenic Acid /Supplemented Diets Prevents Serum Arachidonic Acid Accumulation in Humans. J of Nutrition, 2000;130: 1925-1931).

One aspect of the invention is a mixture comprising two components, one rich in EPA and one rich in GLA. The first component is a mixture comprising EPA and DHA comprising at least 35% EPA, preferably 50% EPA and more preferably 60% or more of EPA, and less than 25% DHA, preferably less than 10% DHA. The second component comprises at least 20% GLA, preferably 60%, and more preferably 70% GLA. The preferred composition comprises about 80% of the first component and 20% of the second component. The proportion of the first and second components to needed to achieve the same ratio of EPA:DHA:GLA as in the preferred composition may be achieved from other sources of EPA, DHA, and GLA. Note that first component is enriched in EPA as compared to the concentration in which it normally occurs in Fish Oil and the second component is enriched in GLA compared to the most concentrated natural source, borage oil which is about 18% GLA. The mixture preferably further comprises one or more antioxidants to stabilize the composition, such as Rosemary, vitamin E, asthaxanthine, carnitine, ascorbyl palmitate, tocopherols or other antioxidants known in the art for stabilizing fish oil.

The composition may also be used as a substitute for vegetable oil, for instance as a salad dressing or for cooking or baking, particularly if the composition is stabilized with antioxidants. The oil can be incorporated into breads, pizza crusts and baking goods at temperatures up to 350° F. over a 30-minute period. Replacement of vegetable oil in the diet is important because the typical person ingests too much vegetable oils (omega-6) compared to omega-3 oils, which results in production of excessive amounts of pro-inflammatory eicosanoids.

### BRIEF DESCRIPTION OF THE DRAWINGS

These and other features, aspects and advantages of the present invention will become better understood with regard to the following description, appended claims and accompanying drawings, where:

FIG. 1 is a flow diagram of the metabolic pathways for metabolism of Omega-3 and Omega-6 Fatty Acids.

FIG. 2 is a figure showing several products made with composition of the invention.

### DESCRIPTION OF THE PREFERRED EMBODIMENTS

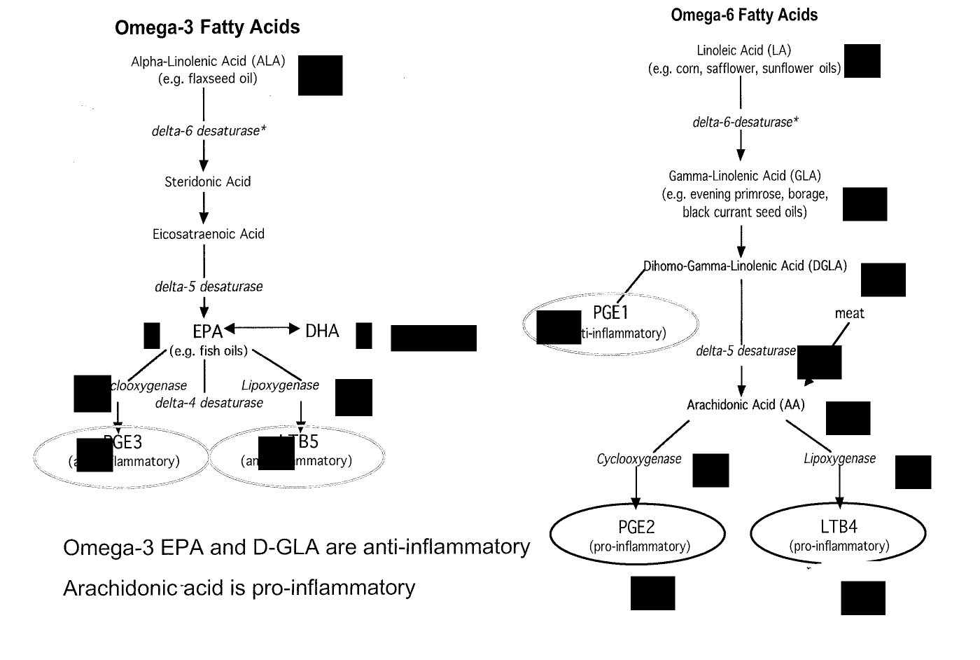

FIG. 1 shows the metabolic pathways for metabolism of Omega-3 and Omega-6 Fatty Acids. Omega-3 Fatty Acids can be found in flaxseed oil in the form of Alpha-Linolenic Acid (ALA) 1. ALA from flaxseed oil is eventually metabolized to EPA 2. EPA 2 and DHA 3 are also principal ingredients of Fish Oils 4. Enzymes cyclooxygenase (COX) 5 and lipoxygenase (LOX) 6 act on EPA to make anti-inflammatory prostaglandins, shown as PGE3 7, and leukotrienes, shown as LTB5 8.

Omega-6 Fatty Acids are found in vegetables such, as corn, safflower, and sunflower oils, as Linoleic Acid (LA) 9. LA is catalytically converted to Gamma-Linolenic Acid (GLA) 10. GLA 10 is also found as an ingredient in certain oils, particularly evening primrose oil, borage oil, and black currant seed oil. GLA is converted to Dihomo-Gamma-Linolenic Acid (DGLA) 11. Anti-inflammatory prostaglandin PGE1 112 is formed from DGLA. DGLA is also acted on by enzyme delta-5 desaturase 13 to form Arachidonic Acid (AA) 14. AA is acted on by COX 5 and LOX 6 enzymes to form pro-inflammatory prostaglandins, shown as PGE2 15, and pro-inflammatory leukotrienes, shown as LTB4 16.

It is known that addition of eicosapentaenoic acid (EPA) to GLA inhibits delta-5-desaturase activity, the terminal enzymatic step in AA synthesis. (Barham J B, Edens M B, et al., Addition of Eicosapentaenoic Acid to Gamma-Linolenic Acid/Supplemented Diets Prevents Serum Arachidonic Acid Accumulation in Humans, J of Nutrition, 2000;130 :1925-1931). Thus less AA is formed than would have been in the absence of EPA. Also in the presence of both EPA and AA, both compete for the COX and LOX enzymes to make their anti-inflammatory and pro-inflammatory prostaglandins and leukotrienes, respectively.

The preferred embodiments of the invention concentrate EPA and GLA to levels greater than their concentration in fish oil and GLA containing vegetable oils, respectively, and combines them into a single composition. A preferred embodiment comprises a mixture comprising two components, a first component rich in EPA and a second component rich in GLA. The first component is a mixture comprising EPA and optionally DHA comprising at least 35% EPA, preferably 50% EPA and more preferably 60% or more of EPA, and less than 25% DHA, preferably less than 10% DHA, possibly 0% DHA. The second component comprises at least 20% GLA, preferably 60%, and more preferably 70% GLA. The preferred composition comprises between about 60 and 80% of the first component and 20% and 40% of the second component. The proportion of the first and second components to needed to achieve the same ratio of EPA:DHA:GLA as in the preferred compositions may be achieved from other sources of EPA, DHA, and GLA in place of the two components. Note that first component is enriched in EPA as compared to the concentration in which it normally occurs in Fish Oil and the second component is enriched in GLA compared to the most concentrated natural source, borage oil which is about 18% GLA. The mixture preferably further comprises one or more antioxidants to stabilize the composition, such as Rosemary, vitamin E, asthaxanthine, carnitine, ascorbyl palmitate, tocopherols or other antioxidants well known to those skilled in the art for stabilizing fish oil.

The first component containing 35-60% or more of EPA and less than 25% DHA can be concentrated by multi-stage molecular distillation of fish oil, such as cod liver oil or more preferably body oils from sardines or anchovies, which start out richer in EPA. The oil is preferably filtered through bentonite clay to reduce impurities. The second component may be made by distillation of a vegetable oil such as borage seed oil or primrose oil, preferably borage seed oil. Alternatively GLA may be concentrated using supercritical CO₂ extraction or fractional crystallization borage seed oil as are well known to those skilled in the art. Both concentrated EPA (60%) and GLA (90%) are available in pharmaceutical commerce.

In principal, the best anti-inflammatory results are obtained using as pure as possible EPA and GLA in the two components; however both components are more expensive when most concentrated. So it is possible to balance degree of anti-inflammatory efficacy against cost.

The invention is valuable as a pharmaceutical or nutritional supplement for reducing inflammation in the body (as an NSAID), dilating blood vessels, reducing pain, relaxing muscle spasms, reducing blood clotting, lowering harmful cholesterol levels and raising beneficial cholesterol levels, reducing blood triglyceride levels and blood pressure, increasing immune functions and visual acuity, and improving the nervous system. All of the anti-inflammatory benefits of omega-3 fatty acids should be realized in a more efficacious way.

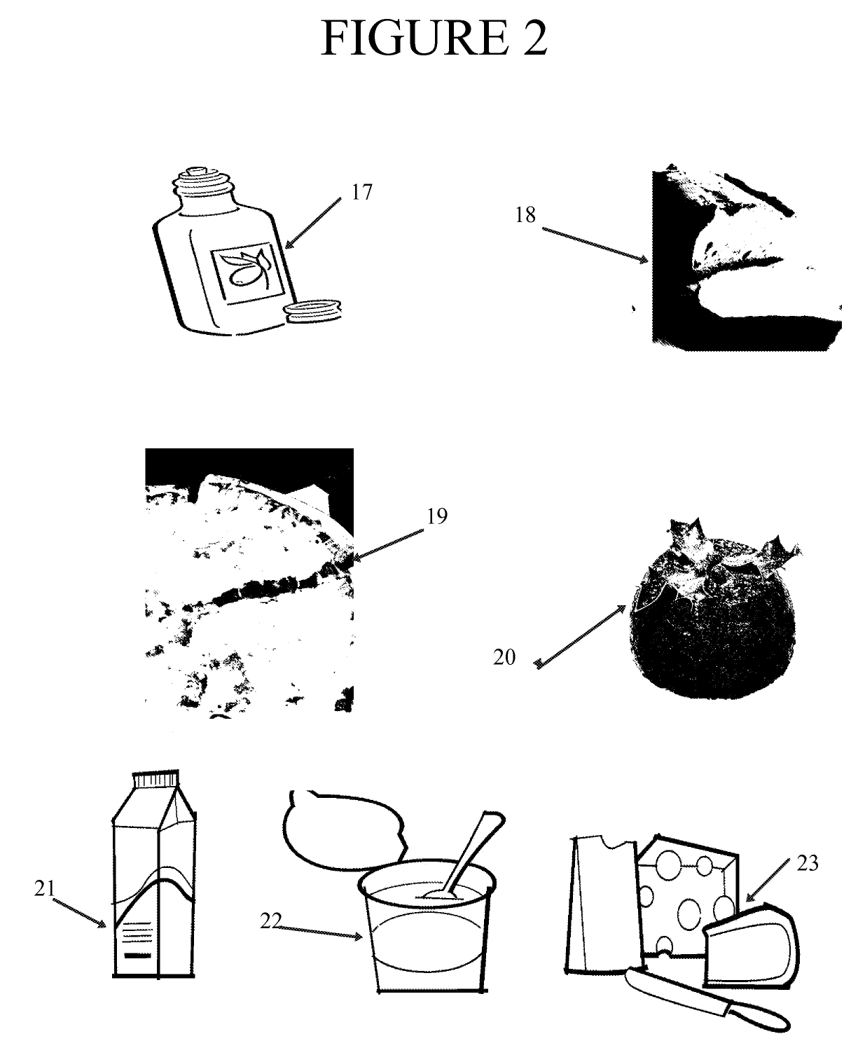

Referring to FIG. 2, the composition may also be used as a substitute for vegetable oil 17, for instance as a salad dressing or for cooking or baking, particularly if the composition is stabilized with antioxidants. The oil can be incorporated into breads 18, pizza crusts 19 and baking goods 20 at temperatures up to 350° F. over a 30-minute period. Replacement of vegetable oil in the diet is important because the typical person ingests too much vegetable oils (omega-6) compared to omega-3 oils, which results in production of excessive amounts of pro-inflammatory eicosanoids.

The composition can be incorporated into milk 21, yoghurt 22, and cheese 23, in place of milk fat.

The product may be dispensed as a liquid or a gelatin capsule. The adult dosage is 500 to 2000 mg./day.

Although the present invention has been described in considerable detail with reference to certain preferred versions thereof, other versions are possible. Therefore the spirit and scope of the appended claims should not be limited to the preferred versions herein but only to the appended claims.

What is claimed is:

1. An anti-inflammatory composition comprising between about 60% and 80% of a first component, said first component comprising greater than 35% of EPA and comprising less than 25% of DHA, and 20% to 40% of a second component, said second component comprising greater than 20% GLA.

2. The composition of claim 1, wherein the first component comprises greater than 50% of EPA.

3. The composition of claim 1, wherein the first component comprises greater than 60% of EPA.

4. The composition of claim 1, wherein the first component comprises less than 10% of DHA.

5. The composition of claim 1, wherein the first component comprises less than 1% of DHA.

6. The composition of claim 1, wherein the second component comprises greater than 60% GLA

7. The composition of claim 1, wherein the second component comprises greater than 69% GLA

8. The composition of claim 1, further comprising an anti-oxidant.

9. The composition of claim 3, wherein the second component comprises greater than 60% GLA.

10. The composition of claim 3, wherein the second component comprises greater than 69% GLA.

11. The composition of claim 9, further comprising an anti-oxidant.

12. The composition of claim 10, further comprising an anti-oxidant.

13. A product comprising the composition of claim 11 incorporated within is a human food.

14. The product of claim 13, wherein the food comprises salad dressing.

15. The product of claim 13, wherein the food comprises a dairy product.

16. The product of claim 15, wherein the dairy product comprises milk.

17. The product of claim 15, wherein the dairy product comprises cheese.

18. The product of claim 15, wherein the dairy product comprises yoghurt.

19. The product of claim 13, wherein the food comprises a baked good.

20. An anti-inflammatory composition comprising at least 20% EPA and at least 6% GLA.

21. The anti-inflammatory composition of claim 20 comprising at least 45% EPA and at least 12% GLA.

22. The anti-inflammatory composition of claim 20 comprising at least 50% EPA and at least 15% GLA.
